(19) United States
(12) Patent Application Publication
(10) Pub. No.: US 2012/0180180 A1
(43) Pub. Date: Jul. 19, 2012

(54) Seeing aid or other sensory aid or interface for activities such as electric arc welding

(76) Inventors: Mann Steve, Toronto (CA); Mir Adnan Ali, London (CA)

(21) Appl. No.: 13/329,210

(22) Filed: Dec. 16, 2011

(30) Foreign Application Priority Data
Dec. 16, 2010 (CA) 10-12-1488

(51) Int. Cl. A61F 9/06; F21V 33/00; H04N 7/18; B23K 9/00

(52) U.S. Cl. 2/12; 219/136; 362/33; 348/61; 348/E07.085

(57) ABSTRACT

A method, means, apparatus, and system is disclosed, for use with an electrically controllable light-producing activity, or possibly an electrically-controlled sound producing activity. In one embodiment, the light is modulated to affect an imaging function, as a secondary effect, without substantially affecting a primary or main purpose of the light-producing activity. In another embodiment, the light is modulated to affect an imaging function, as a secondary effect, in conjunction with effects on the primary or main purpose of the light-producing activity. The invention is useful, for example, in TIG (Tungsten Inert Gas) welding where the light-producing arc, and possibly some light-producing utility lights, are modulated to improve a computer vision system (such as an auto darkening welding helmet or a headup display for a welding helmet) that helps a person see better. Cybernetic and physiological sensing is disclosed such as EEG, ECG, and the like, in conjunction with wearable computing.

This application claims priority to Canadian Application No. 10-12-1488 Filed 2010 Dec. 16, the entire disclosure of which is incorporated by reference.

### FIELD OF THE INVENTION

The present invention pertains generally to new kinds of imaging technologies, seeing aids, control systems, and the like, which assist a person engaging in a light-producing activity such as electric arc welding, or other multimedia light or sound-generating activities, or situations, potentially of extreme dynamic range.

### BACKGROUND OF THE INVENTION

Certain activities, by their very nature, produce sound, light, or other perceptible disturbances which make it difficult to perceive clearly their effects, and the like. For example, arc welding produces a bright light that makes it necessary or desirable to wear protective eyewear which at the same time makes it harder to see clearly.

### BRIEF DESCRIPTION OF THE DRAWINGS

The invention will now be described in more detail, by way of examples which in no way are meant to limit the scope of the invention, but, rather, these examples will serve to illustrate the invention with reference to the accompanying drawings, in which:

FIG. 1 depicts a computer vision and multimedia system architecture for performing light-producing tasks, and the like.

FIG. 2 depicts a welding helmet incorporating aspects of the invention.

FIG. 3 depicts a block diagram of the system.

FIG. 4 depicts signal processing of the system.

FIG. 5 depicts incremental image processing, in which a combined signal is updated each time new information arrives.

FIG. 6 depicts signal processing of two differently exposed images of approximately the same subject matter, using an algorithm of the electric seeing aid invention.

### DETAILED DESCRIPTION OF THE DRAWINGS

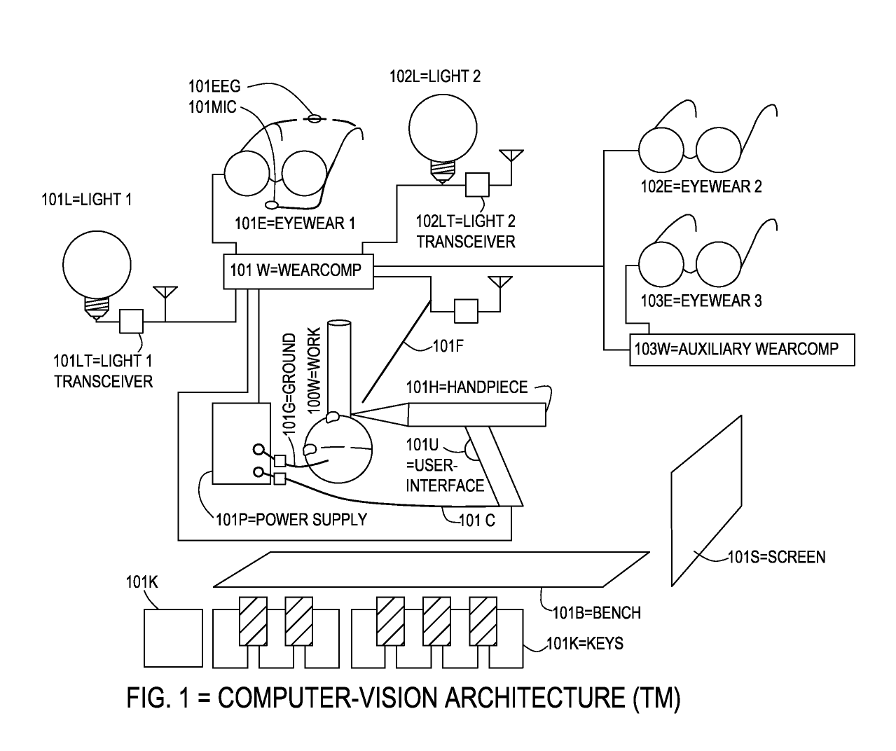

FIG. 1 depicts an example of the Computer Vision ARChitecture™ of the invention. A wearcomp 101W (wearable computer) exists on the body or bodies of one or more users of the invention, or a wearcomp 101W exists on a first user and a wearcomp 103W exists on one or more other users or observers or assistants in the immediate vicinity or at one or more remote geographic locations.

A piece of work 100W is depicted in the drawing as a hydraulophone pipe having a spherical bulb connected to a cylindrical neck, but any other piece or pieces of work may be understood to be present.

The work is connected to power supply 101P by a heavy ground clip with a heavy flexible wire, such as American Wire Gauge (AWG) 000, denoted as ground 101G.

A handpiece such as a MIG torch or TIG torch, soldering iron, drill, plasma cutter, saw, or other device is denoted as handpiece 101H. The handpiece has associated with it one or more user interfaces such as user interface 101U which may be a squeeze trigger on the handpiece or may be a separate foot pedal, or separate “deadman switch” or separate device or devices such as an eye tracker, an electrocardiogram (ECG) device, or brainwave sensor, i.e. an electroencephalogram (EEG) device incorporated into eyewear 101E. Typically the EEG sensor resides in an eyeglass safety strap at the back of the head, or in the headband of a welding helmet, or the like, as for example, occipital lobe sensor 101EEG. Occipital lobe sensor 101EEG measures brainwave activity and uses this to control the welding parameters. The simplest embodiment of this “Mind Over Metal”™ aspect of the invention is to simply amplify the raw brainwaves and use this as the welding output. This can be fun and interesting in the sense that one is using one's mind to melt the metal, as a sort of “mind melding” with molten metal. A more useful, perhaps, embodiment of this aspect of the invention realizes that there is a lot of useful information that takes place in the subconscious mind during the welding process, and that when one is really in the “zone” of top mental performance, one has a high Beta and Alpha wave content at the same time. Thus a cybernetic brainwave controlled welding system is useful. Another example application is to use a wearable microphone 101MIC for voice command of the wearable computer, i.e. for voice-controlled welding. But an even better embodiment is to simply be able to sing a note into the microphone and have the welding current mimic that note for welding aluminum, for example. A pitch detector thus sets the weld frequency. Alternatively, the raw audio from the microphone is simply amplified, and fed to the welding circuit. This allows the user to control the frequency AND waveshape to some degree, i.e. through changes in timbre of the voice, as well as impart tremelo and vibrato, as well as dynamics (e.g. sing a note louder to increase the weld current), etc. As pertaining to a sense of pitch (musical notes), there may also be a sense of rhythm and tempo, such as, for example, may be embodied through an electrocardiogram (ECG). Rhythm and tempo are important to, for example, applying filler rod to the puddle of a weld, and rather than continuously applying filler, it is often desired to periodically touch the filler to the puddle. This can be done at a certain rate. An awareness of one's own physiological state can help in this regard, as for example, when the power supply 101P is responsive to an output of an ECG device. In one embodiment, the ECG device controls the pulse frequency of power supply 101P. In another embodiment, the ECG device controls an attribute of a singing TIG welding arc, to signal a steady tempo. In another embodiment, there is a sensor that senses travel speed of a tungsten electrode, and signals to the user when to apply filler rod, in order to maintain a steady spatial frequency. The spatial frequency is preferably compared with temporal frequency and there is a feedback system to match this difference to the natural physiological responses of the user.

The appratus of FIG. 1 is useful in processes that cause the generation of visible light. Examples of such processes include photography, photographic “lightpainting”, lightvector-painting, augmented reality, mediated reality, grinding, brazing, welding, and the like.

By way of example, user-interface 101U might be a squeeze trigger on handpiece 101H, such that squeezing the trigger harder increases the current supplied in a welding process.

Typically user-interface 101U is connected to power supply 101P by a cable such as cable 101C comprising a heavy wire and perhaps one or more lightweight wires or wireless communications to pass information to and from user-interface 101U to and from power supply 101P. Alternatively, the informatic connection may be separate from the power to handpiece 101H, as, for example, might be the case when user-interface 101U is a foot pedal with its own cord or wireless connection.

In this situation, eyewear 101E might take the form of an automatic darkening welding helmet which might also bear one or more wearable cameras, imaging systems, and aremacs such as projectors that project from the helmet onto work 100W and a nearby workbench or the like. Eyewear such as eyewear 101E, 102E, etc., is assumed to mean a helmet or eyeglasses or even a handheld display that can be looked at or looked through.

Eyewear 101E may be an augmented-reality, virtual-reality, or mediated-reality device or may include such devices. For example, a welding helmet may include a headup display upon which may be displayed information and views of the weld process in alternate regions of the invisible light spectrum, or the like.

Work 100W is illuminated with ambient room light, sunlight, or various forms of controlled and artificial light. A utility light 101L is connected informatically to Wearcomp 101W by a wireless light transceiver 101LT, or by a direct physical connection, or by sending data over powerlines, or by any of a variety of other means.

In one embodiment of the invention, the handpiece 101H produces a modulated output that works in concert with the light 101L. When welding aluminum the modulation of handpiece 101H occurs naturally by the Alternating Current (AC) used in welding aluminum, but when welding steel or stainless steel which is ordinarily done with Direct Current (DC), the DC may be strongly pulsed or otherwise modulated to affect the weld, or weakly pulsed in such a way as to not affect the weld, but simply to affect the computer-mediated reality in which eyewear 101E operates.

By mediated-reality I mean also augmented-reality and virtual-reality which are both proper subsets of mediated-reality.

In one mode of operation wearcomp 101W works with eyewear 101E to capture a High Dynamic Range (HDR) image of work 100W or whatever else the wearer or user might be looking at. Typically Wearcomp 101W captures images from one or more helment-mounted cameras on the helmet eyewear 101E. Typically different exposures are captured, per frame.

Wearcomp 101W issues commands to pulse light 101L and also either monitors power supply 101P or handpiece 101H or commands power supply 101P or handpiece 101H such as to capture an image frame at a time when the light output from handpiece 101H is strongest, and at a time when the light output from handpiece 101H is weakest.

More generally, a plurality of images due primarily to changes in light output of handpiece 101H are captured, to define a lightvector that is due primarily to the light from handpiece 101H. The concept of lightvectors in general, is well known, as, for example described in chapters 5 and 6 of the textbook “Intelligent Image Processing” published by John Wiley and Sons, author S. Mann, ISBN 0-471-40637-6. Let us call the lightvector due to the light from handpiece 101H vector Vₕ. The lightvector Vₕ may be captured at very high dynamic range, by capturing various differently exposed images due to the lightvector, and also by using a lock-in camera. A lock-in camera is defined as a camera in which each pixel of the camera is or can be worked as a measuring device, to be sensitive to light primarily from a particular source that is modulated in a particular way.

In welding aluminum, the AC signal supplied by the welding power supply 101P is the signal that the lock-in camera locks into. When welding steel, a DC signal is superimposed with a spread spectrum or tone-based signal or other information-bearing signal which will not affect the weld, but will be such as to produce a coded light source that the camera can selectively tune to, based on the ability of the lock-in camera to ignore other light sources and pay particular attention only to the light due to the handpiece 101H.

Likewise the wearcomp 101W issues commands to one or more utility lights such as lights 101L, 102L, etc. In this way the lock-in camera can be made to be particularly sensitive to the light sources. For example, a lightspace (set of images each due to a particular light source) is captured. One image as the scene would appear under only the light source from handpiece 101H is captured. This image, as lightvector Vₕ is preferably captured due to various levels of the light source as a high dynamic range (HDR) image of how the scene appears when illuminated only by the torch light of handpiece 101H.

This lightvector Vₕ can be determined comparametrically, or with a camera that reads out in linear quantimetric units.

Another lightvector V₁ is the lightvector due to light source 101L. Lightvector V₁ is, or is representative of, is approximately, or is approximately representative of, an image ƒ₁ of how the scene would look if it were only illuminated by light source 101L.

Likewise another lightvector V₂ is captured of the scene as if illuminated only by lightsource 102L and nothing else.

An ambient lightvector, V₀, is captured of how the scene would have looked were it not for the light sources from lights 101L, 102L, etc., and handpiece 101H.

Alternatively these lightvectors may be captured as a lightspace, either superposimetrically (from a superposigram for example), or as linear combinations in activating each light source (lights 101L, etc., and handpiece 101H) with a known sequence. In a simplest embodiment of the invention, the lights may simply be activated sequentially, and an image mostly due to each light may be captured. Then a process of lightvector amplification such as that described in the “Intelligent Image Processing” textbook is applied.

A screen 101S screens off the work area, i.e. blocks view of the work area to onlookers so that they do not experience arc flash, eye damage, or the like. The screen 101S is also a display screen such as a TeleVision (TV) screen, or projection screen, or the like. This screen displays a high dynamic range image of the eyewear 101E, so that onlookers can see what is happening beyond the screen 101S. Screen 101S may also include one or more cameras or other tracking devices that determine locations of various people viewing the screen 101S and render a coordinate stabilized view of the subject matter is it might appear in the absence of screen 101S. Thus screen 101S is a reality mediator that facilitates spectator participation in the welding booth, without the spectators needing to wear welding helmets. The absence of the welding helmets for the spectators allows them to see over a much greater dynamic range of what's happening in a welding booth or other similar space in which a wearer of eyewear 101E is located.

Other screens at the local area, or at other geographic locations allow others to see from a distance into the welding booth at the present time, or in the future looking back. For example, when a hydraulophone pipe of work 100W fails or begins to leak, we can look back and review the time back when the weld was made, and determine why there might have been a problem.

This might also be useful in nuclear reactor parts or offshore oil rigging equipment, parts uses in aerospace, and the like, where weld failure or part failure can be of grave consequence.

Additionally, other users of eyewear such as eyewear 102E, 103E, etc., may remotely participate through wireless or wired link to and from one or more auxiliary wearable computer systems or other processors such as Auxiliary Wearcomp 103W.

While wearable computers are described here, the invention can also be used with fixed cameras such as tripod mounted cameras.

For example, two Flea3 computer vision cameras mounted on either side of a Kinect™ 3d camera system can be tripod mounted to capture the happenings in the welding booth and display this information to the person doing the welding and other persons locally or remotely.

Additionally a torch-mounted camera in handpiece 101H helps the user see the world from the torch's perspective, while, for example, welding together a tight rank of hydraulophone organ pipes closely packed together, where the user is reaching in behind some pipes. Welding pipes with the torch-mounted computer vision system allows the computer to also analyze the pipe weld process and feed back information into a headup display in the welding helmet which is eyewear 101E, or the like.

A smart workbench 101B can be a grounding surface as well as a general purpose interactive smart station.

Light sources 101L, 102L, etc., may also be projectors that lock-in to the eyewear 101E. For example, a projector is made to seem more than fifty thousand times brighter than it would normally appear, if the projector is gated to the helmet. By momentarily shutting of handpiece 101H and turning on a flash of light in the projector such as light 101L, and gating the helmet to let light in, the light 101L illuminates the scene on bench 101B, at the exact instant the eyewear 101E becomes transparent.

For example, let us consider the situation where eyewear 101E is merely a standard auto darkening helmet. For a very brief time period such as one microsecond, the helmet becomes undarkened, while the light source is made to flash strongly. Consider if light source 101L is a xenon strobe flashlamp or projector with strobe flashlamp inside it as the light source.

A computer such as Wearcomp 101W issues a command to light source 101L to flash 100 times each second for a duration of a millionth of a second duration of each flash. The computer also issues a command to an auto darkening helmet to undarken during that time interval. So the helmet undarkens to let the wearer see the scene as illuminated by the projector of light 101L, but the helmet is only undarkened one ten thousandth of the time. Thus the helmet lets in only 0.01 percent of the light incident upon it.

In this way the helmet remains dark enough to weld by, but as if magically allowing itself to be transparent to the light source 101L.

More generally, the helmet might move between a dark state with transmission coefficient c_{d}, and a light state with transmission coefficient cₗ, and we might, for example, have that c_{d}= 1/100,000 and cₗ= 1/10, and then the average transmission

$$c = \frac{t}{T} {c}_{l} + \frac{T - t}{T} {c}_{d} , \qquad ( 1 )$$

where T is the period (e.g. 1/100 sec) and t is the time duration of the flash, e.g. 1/1,000,000 sec). In the foregoing example, therefore, the average transmission coefficient, c, is 1.9999e-05, i.e. the helmet lets through only 1/50,003th of the amount of light from the torch of handpiece 101H.

This attenuation of approximately fifty thousand times, is suitable for TIG welding or the like (i.e. approximately Shade 12), while being only an attenuation of ten times (i.e. only about the same attenuation as typical sunglasses) to the light from the data projector of light 101L.

In this way, a data projector can provide useful overlays on top of the smart countertop or desktop or other surface such as workbench 101B, and be visible while TIG welding.

Thus not only does the apparatus of the invention make the weld itself visible but it also makes the work visible and annotations of the work visible to the user of the apparatus.

The foregoing example is a simple one using a square wave signal fed to an ordinary auto darkening helmet and light source (i.e. synchronization of a utility light with the helmet), but a more advanced system can be made using a specially prepared signal fed to a computer system so that it can be specially time-division multiplex coded as being visible to certain people in a shared workspace. In the foregoing example we have available to us n=t/T=10,000 different time-division multiplex channels, so that up to 10,000 different people in the same place could see different annotation placed on the workbench 101B.

While we don't normally need to have that many users sharing the space, we certainly would often like to have several people in the same space being able to see different information overlaid on top of physical reality.

As another example, consider a large event where everyone wears special glasses. Such events as 3d movies for example, can use the invention. Each audience member may be supplied with information specific to them overlaid onto physical reality such as the hallways and posters in the hallways on the way into the movie theatre. The person can put on their glasses and see something special for them.

More generally, though, if we use a mediated reality where the world is seen not merely through auto darkening helmets or the like, but additionally or alternatively through a camera system, things can get even better.

In one embodiment a user looks through a camera system where the cameras are lock-in cameras, and we can use one spreading sequence for the left eye and another for the right eye, and yet other sequences for other people, etc.

The world of welding or anything else for that matter becomes drawable in computer-mediated reality, as follows:
Activate light source 101L with a spreading sequence to which sensor such as eyeglasses 101E is made sensitive to (e.g. by time-division multiplexing, code-division multiplexing, or the like);
Render data in coordinates stabilized to spatial coordinates of the user of eyeglasses 101E, for this activation;
Activate light source 101L with a spreading sequence to which a different sensor such as another eye sensor of eyeglasses 101E, or another user of eyeglasses 102E, or the like, is made sensitive;
Render data in coordinates stabilized to spatial coordinates of the alternate sensor;
etc. . . .

As can be seen, the invention allows each eye of each user to be supplied with unique information and views and illuminations, and even the main room lights in a large factory could be made to throb and project different material unique for each user, onto their workbench without bothering the other users.

Without the invention it is hard to see clearly. For example, if I use a really bright worklight on my desk, it makes my helmet darken, and so I have to reduce the sensitivity and risk arc flash or need to turn down the work light.

In one aspect of my invention I have a TIG pedal that has a switch in it that simply turns on a worklight only when I'm stepping down on the pedal. The pedal plugs into a special box that has some electrical sockets on it and there's a multipole relay in the box that turns on when I step on the pedal, and the relay turns on the worklights and also turns on the part of the welder originally turned on by the pedal. This little control box can be sold as an add-on to any TIG welder.

In a better embodiment, the little box turns on a strobolux and strobolume flashing light, that is gen-locked to my helmet, so I can see as if the light is some five thousand times brighter than it would otherwise be. Thus when I step down on the pedal, a double pole relay turns on the strobe worklight and the second pole of the relay closes the contacts that the foot pedal ordinarily closes on the welder.

Then what I see with the 100 watt or so light is as if the light is outputting five hundred thousand (500,000) watts, i.e. I can see it when the helmet has darkened, and therefore I can see my work really clearly and I can look around the room and see everything in the room really clearly when the helmet is dark.

That way I can weld up a big hydraulophone sculpture and see all the other organ pipes, not just the one I'm welding just near where the weld is being illuminated by the light from the torch.

Additionally, I can sequence different utility lights and therefore adjust the lightspace to see as if the brightness of each of the lights can change after a video recording is made, e.g. I can go back and look at welds I did before, and see how it would have looked in the dark, and then decide to see how it would have looked left-lit, and then decide to see how it would have looked right-lit.

Being able to retroactively change the shadows makes it easier to see what happened in the past.

In another embodiment of the invention, a smart countertop or desktop or other space such as a workbench 101B is fitted with a plurality of sensors and effectors linked with the illumination process, etc. Work bench 101B is a workstation which can be surrounded on 1 or more sides with a shroud formed by screens such as screen 101S. In one configuration, the bench 101B is surrounded on 3 sides with three screens 101S, to prevent anyone from seeing the bright light on the bench, except for the one or more people with protective eyewear working with handpiece 101H. Various imaging systems including headworn cameras or bench mounted cameras capture the subject matter on bench 101B and the surrounding environment. The lightspace and high dynamic range images are brought together in a three-dimensional environment, and then rendered to the three screens, such that each of the three screens presents one of a front, left, and right side view. In this way, others in the room can see what's on the bench as they walk around and look at the bench from various angles. The previously mentioned tracking devices can be applied to each of the three screens independently or together.

The aspects of the invention depicted in FIG. 1 are useful for any of a wide variety of light-producing tasks such as photographic “lightpainting”, metalwork, plasma cutting, stick welding, MIG (Metal Inert Gas) welding, and TIG welding. In, for example, TIG welding, there is a relatively high degree of coordination among the various body parts of people who do TIG welding. For example, most people performing this art are skilled in the coordination of both hands and also with the foot pedal. While holding a handpiece 101H in one hand, they can also coordinate another object, such as a filler rod 101F, in their other hand, while, at the same time, skillfully operating a foot pedal.

This ability to coordinate these 3 tasks at the same time, is analogous to the way an organist can easily coordinate various parts of music with both hands, and feet, playing one “manaul” (keyboard) with the left hand, and a different manual with the right hand, and at the same time playing another part on the pedal division, which itself resembles a keyboard, and has the white and black foot pedals laid out much like a piano with giant “foot sized” keys.

Indeed, there is a lot of similarity between TIG welding and the organ, as both involve a great deal of artistry and creativity.

The traditional foot pedal on a TIG welder adjusts the current flow to the handpiece 101H. In this sense, it is analogous to the “volume pedal” or “swell pedal” of the organ, in the sense that it controls the output amplitude of power supply 101P.

Within the context of the present invention, there is provided means for nuanced and careful control of the welder, by way of a better pedal or pedal-like control, in which more parameters of the welder power supply 101P or the process in general can be controlled.

In one embodiment there is an array of pedal keys 101K, which can be arranged like the effects pedals used by a guitarist, or like the keys on an organ pedalboard, for example. The 12 black and white keys shown correspond to various musical pitches, which can, for example, be used for welding aluminum, and the leftmost key 100K corresponds to Direct Current (DC), which can, for example, be used for welding steel.

When welding aluminum, for example, Alternating Current (AC) is used, typically, although there may remain some DC offset. With a welder power supply 101P, the frequency of the AC can be selected. High frequencies tend to focus better in some areas and in other areas, less focus is desired, i.e. low frequencies are better.

Consider the situation of welding a thick to a thin piece of aluminum. For example, when a rigid thick piece of aluminum crossbar is being welded across the opening of an aluminum sheet metal electrical box made of thin material, there is a boundary between thick and thin material.

Often one finds oneself adjusting the frequency on-the-fly, reaching over to the power supply 101P to set a frequency control, and moving this up and down, or having to settle for a frequency that's neither ideal for the thick nor the thin, but somewhere in between.

In one embodiment, the frequency and amplitude may be controlled together, such that the frequency goes up when the amplitude goes down, so that one can ride the volume pedal up and down and also control the frequency to get better focus with high frequency at low currents on the thin material and better spread with low frequency at high currents on the thick material. This embodiment is achieved by way of a LookUp Table (LUT) that selects a frequency from a list of amplitude values, e.g. 55 cycles per second if the amperage is less than 50, and 110 cycles per second for amperages between 50 and 100, and 220 cycles per second above 100 Amperes. This can also be made more continuous, but since the AC welding makes a loud sound, it is nicer if it makes a musical sound and if the pitch changes in musical intervals that are easier for a human to hear and understand and become attune to.

Many people doing welding like “death metal” so the notes could even to be tuned to a Locrian mode (i.e. corresponding to the white keys of the piano going from B to B), but others may prefer a natural minor scale (i.e. corresponding to the white keys of the piano from A to A).

In this way, the pitch can be heard clearly.

Indeed, one aspect of the invention is to use the welding process as a plasmaphone or ionophone to provide some multimedia aural feedback to the user. Pitch changes in the welding supply 101P can thus be used to convey important information to the person using it.

In another embodiment, a computer vision camera looking at the welding line, helps a person stay on the line and keep a straight line, by making a tone that changes in pitch as the line is deviated from. For example, being on the line with the torch in close gives back a high pitched tone, and as the user deviates the pitch drops, which also protects the weld by spreading the beam and reducing the concentration of energy.

In another embodiment, this aural feedback comprises a warning tone that can even be a musical chord. For example, we can create any arbitrary waveform with power supply 101P. In one embodiment, a major chord is sounded to signify everything is going well. The power supply 101P changes the output chord to minor to warn the user that there is a potential or eminent problem, or to be careful.

This feature of the invention makes the welder's life almost as if life had a soundtrack. In a movie, we often imagine we're the actor or the hero ourselves. We know when we hear a minor chord, we need to be careful, i.e. maybe there's someone hiding around the next corner pointing a gun at us.

Likewise, when using the invention, the user feels as if they are in a movie that has a soundtrack, and they can listen to the sounds made by the welder power supply 101P as it powers and ionophonizes the arc, such that the sounds can be heard.

A useful waveform is a musical chord, comprising of various Fourier components, for example, power supply 101P can generate a waveform that is equivalent to it simultaneously generating the following three frequencies: 220.00 cps, 261.63 cps, and 329.63 cps (Cycles Per Second). The human ear perceives this as a minor chord, in particular, A-minor.

This chord is generated when things are “dangerous” i.e. when the tungsten is getting too close to the puddle, or when conditions warrant extra caution. When things are going smooth and well, the middle frequency of 261.63 cps (“C”) gets changed to 277.18 cps (“C-sharp”).

The sensing of when things are going well or not, is done by a helment mounted camera and a station mounted camera and simple computer vision algorithms. Alternatively the sensing is done by plasmatic means, i.e. sensing of plasma conditions by way of driving point impedance characteristics as sensed by power supply 101P. For example, short-circuit detection triggers production of a minor seventh chord, such as Am7=“A”, “C”, “E” and “G”, but eminent short circuit conditions just trigger a shift to a minor triad “A”, “C”, and “E”.

This gives an ability to convey a range of severities ranging from “powerful” with just “A” and “E” sounded, to major (A, Csharp, E), then minor (A, C, and E), and finally, minor 7th (A, C, E, G).

Audio feedback is useful when arcing different parts of the metal with different “notes” (frequencies).

For example, I'll hit one part of a piece of work 100W with a high “E” and then hit another part with a low “A”, back and forth, heating both parts, to a kind of rhythm that creates good puddle disturbance, and gives rise to a stronger weld, and better penetration on the thick part without blowing through the thin part.

To do this, I use keys 101K, to be able to quickly stomp out different notes into the power supply 101P. With my foot, I can hit one note, and then another, and each is like a separate pedal.

At times, also, I can use both feet to play two notes at once, and get a superposition of two different welding frequencies at the same time.

The leftmost key 100K is a DC key, that is like a key at minus infinity, if the other keys are thought of as logarithmically spaced frequencies.

Alternatively, two pedals can be used, one for pitch, controlled, for example, by the left foot, and a separate pedal for volume, controlled, for example, by the right foot.

Most users of TIG weldering equipment like to put expression into their welds in the way the agitate the puddle which leaves their signature mark. You can often tell who welded something by the way it looks.

Using this embodiment of the invention is like playing a violin, where the user uses the left foot to control the pitch and vibrato (as you'd use your left hand on the violin) and the right foot to control the volume (amperage) and tremelo.

In this description, I use the term “tremelo” to encompass “pulse” or “pulse arc” or the like. Tremelo is the fluctuating volume often used in guitar effects, for example.

Tremelo is amplitude modulation (AM). Frequency modulation (FM) is called vibrato. It is common in musical instruments, but not previously used in welding. Thus some embodiments of this invention bring vibrato to the welding process.

High frequencies focus better in some areas and less focus is needed in other areas, so actually modulating the frequency while circling around in a weld makes a lot of sense in many situations.

The invention thus allows the user to “hit higher notes” on certain areas of the weld while circling around in a pattern that gets a rhythm going, to, for example, bounce back and forth between two or more notes.

This can be done with the two pedals, or with the pedal division that looks similar to the pedalboard on a church organ, or like the array of pedals a guitar player uses.

Making hydraulophones involves a lot of welding thick-to-thin material where the innovative welding technique of this aspect of the invention is very useful.

This system “ARC-hitecture”™ of FIG. 1 includes various sensors, which are also, in some embodiments, connected wirelessly to the welding helmet, so that the vision system in the welding helmet adapts to the specific command from the pedal division or the like (e.g. knowing the nature of the arc can make it easier to see in the encoded vision system, and provides also data for the encoding).

Vision encoding is also adaptive to the “music” being played, i.e. the frame rate of the image capture can be gen-locked to the musical welding. In a simple embodiment, image capture happens at zero crossings of power supply 101P, as well as at maxima. Capturing at zeros and maxima gives a lightspace of weakest to brightest arc, allowing the reconstruction of the arc and non-arc illuminated scenes, which the wearable computer uses to render lightvectors in a high dynamic range lightspace image for presentation in a headup display in the helmet.

For DC welding (e.g. welding steel or stainless steel), we can still pulse the DC to cause it to make sound. Moreover, we can have a superposition of DC and AC that causes the arc to sing, and we can therefore still use this singing arc as a form of aural feedback.

The singing arc aspects of the invention are useful for a variety of different applications. I propose, for example, a “VIOLine”™ system that tracks how a person stays on a line with a torch or the like, and makes a change in sound in the arc to warn of going off the line, or deviating from it.

A similar “TIGline” system produces a feedback control sound in response to the following of a line with a TIG torch and also uses feedback to indicate distance to from the tungsten tip to the metal.

Additionally, a method of doing business in selling products using this invention can comprise the use of the invention as a new musical instrument to help promote the product, or as another product in its own right.

Method of promoting the ARChitecture™ product: creation of a live musical performance with feedback plasmaphone Helmholtz resonators, as follows: A tubular neck feeds into a bulb containing a listening transducer. This sound is fed back into the welder as an amplifier, and this amplified signal goes to the torch. Thus the arc sings, and what it sings is what it “hears” in the bulb.

Therefore due to acoustic feedback the arc sings in resonance to the tune of the bulb and neck.

More generally a plurality of hydraulophone pipes or other similar Helmholtz resonators or other kinds of resonators is used to generate a feedback that depends on which mouth the arc is near.

The invention disclosed here is applicable to robotic welding as well as welding by hand. Without loss of generality, consider, presently, welding by hand, in which case the welding helmet can be used, or also for inspection or supervision of robotic welding the helmet can also be used.

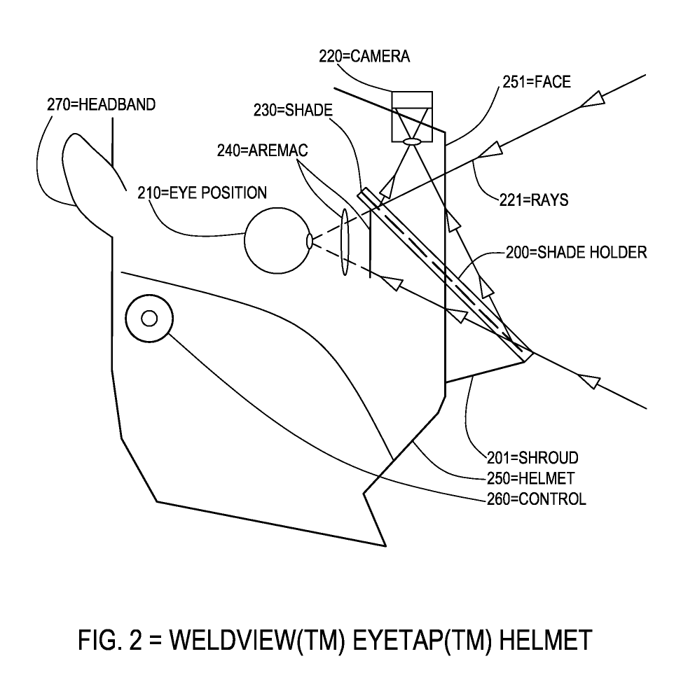

FIG. 2 depicts the welding helmet or eyewear 101E, 102E, or the like. A shade holder 200 holds a first-surface mirrorshade, with the mirror side facing outwards. First surface mirrorshades are commonly used, and available in polycarbonate or glass. Satisfactory mirrorshades include PART #P45811 made by FIBRE METAL (CANADA) LIMITED (a glass SHADE 11), or a GENTEX OMNI VIEW polycarbonate shade, or a ProStar (by Praxair) PRS64219 SHADE 12 Gold Coated Polycarbonate Filter Lens.

The outward facing surface is gold or aluminum. A satisfactory size is 4.5 by 5.25 inches (approx. 114 mm by 133 mm). Normally the shade is mounted in a helmet such as helmet 250. A suitable helmet is the Praxair ProStar helmet. The helmet is modified so that instead of running up and down on face 251 of helmet 250, the shade 230 sits at an approximate 45 degree angle with respect to face 251. In this way, camera 220 looks down and “sees” a mirror image in the reflective outward-facing first surface of shade 230.

The camera 220 is preferably a stereo pair of camera devices, such that it produces a stereo image capturing rays 221 of eyeward bound light that are collinear with rays of light passing through the center of projection of eye position 210. Thus the camera 220 is preferably an EyeTap camera. A left part of camera 220 preferably captures a left-eye signal, and a right part of camera 220 preferably captures a right-eye signal.

The camera 220 sends an output to a processor which then processes the images to display them on aremac 240. Aremac 240 is preferably a stereoscopic display. A satisfactory stereoscopic display is a modified Crystal Eyes™ product manufactured by Microoptical Corporation. The modification is by way of cutting the cord off and driving the left and right eyepiece separately by two separate DCUs (display control units). This can be done by purchase of two Crystal Eyes products and using one pair of eyeglasses with the DCU from that one unit together with the DCU from the other unit. Preferably the processor supplies two NTSC signals, one for the left eye and one for the right eye. A control knob or the like, e.g. control 260 can control the processor to adjust parameters of the processed images for optimal display. A control for headband tension of headband 270 can be incorporated near the control 260 or separately. The headband 270 houses electrodes in contact with the occipital lobe of the wearer to monitor brainwave activity and adjust image content accordingly.

The shade 230 can slide in and out of shade holder 200 so that it can be replaced, e.g. with various transmission coefficients for various tasks. A shroud 201 seals shade holder 200 from stray light.

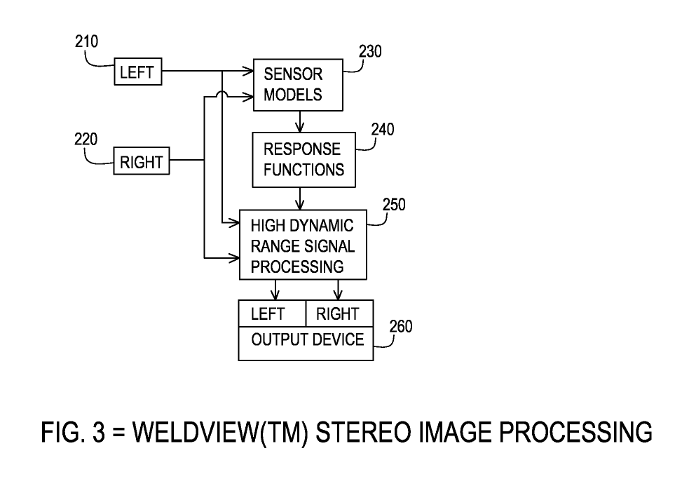

FIG. 3 depicts the processing of the images captured by cameras 210 and 220, for display on the welding helmet or eyewear 101E, 102E, or screen 101S, or for use with robotic welding. In the case of robotic welding, there are embodiments when there is no human intervention, in which case the high dynamic range images are also useful for computer vision and automated guidance of a robotic welding head.

The camera model 230 is updated by sampling the inputs 210 and 220, to adapt to changing conditions. For example, during operation a CCD camera may rise in temperature, altering the camera response function.

This camera model is used to pre-compute useful quantities such as the camera response functions 240 that allow for realtime operation of the signal processing system 250.

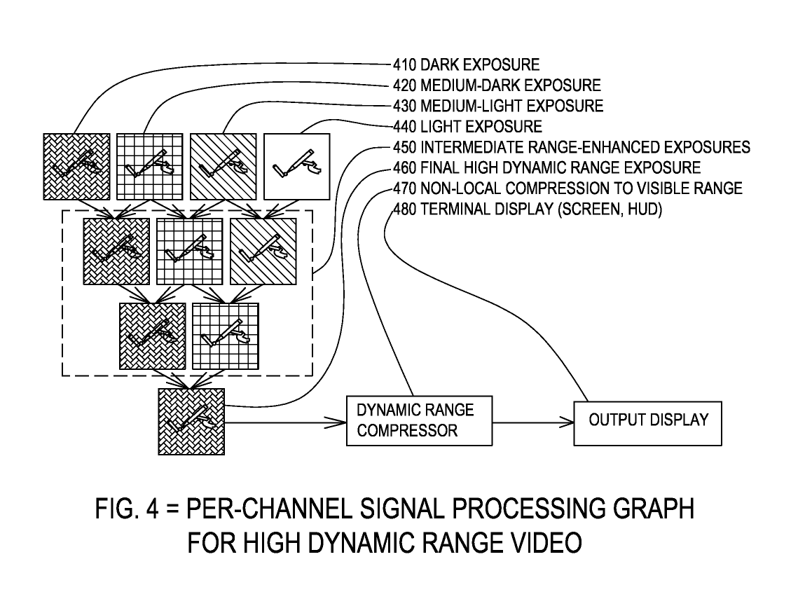

FIG. 4 depicts a fully-connected signal processing graph used to create an High Dynamic Range (HDR) image 460 from four input Low Dynamic Range (LDR) images 410, 420, 430, 440. These input images may be directly obtained from a single CCD camera serially, or from an array of cameras with registered images, where each camera has different exposures (by varying exposure times, sensor array sensitivities, shooting through various different filters, or the like). The useful information from each input image is combined to create a single composite image containing details in the highlights and lowlights of the scene. In this diagram the combining of images is shown as being done pairwise, but in general various embodiments are possible.

The electrically-controlled light-producing equipment is rendered to the operator via an interface that is mediated by the present invention to enable the operator to sense a larger dynamic signal range than is possible using the unaided human sensory apparatus, namely the eyes and ears.

FIG. 4 illustrates the composition of multiple low-dynamic range signals into a single representative high-dynamic range signal with a greater range than any single one of the input signals.

With reference to FIG. 4, mathematically, we denote the contents of 410 as ƒ₁, 420 as ƒ₂, 430 as ƒ₃, 440 as ƒ₄, and 460 as ƒ(\hat{q}) in the following equations.

In this description, let ƒ as a function represents the camera response function (CRF), and as a scalar represent a tonal value, and as a matrix represent a tonal image (e.g. a picture from a camera). We consider a tonal value ƒ to vary linearly with pixel value but on the unit interval, and given an n-bit pixel value υ returned from a physical camera, we use ƒᵢ=(υ+0.5)/2ⁿ, where we have N images, iε{1, . . . , N}, and each image has exposure kᵢ. The subscript indicates it is the i-th in a Wyckoff set, i.e. a set of exposures of the same subject matter differing only in exposure, and by convention kᵢ<kᵢ₊₁∀i<N. The notation for the inverse of the CRF, ƒ⁻¹, means the mathematical inverse of ƒ if it has only one argument, and otherwise means a joint estimator of photoquantity, q.

Camera output is modeled as ƒᵢ=ƒ(kᵢq(x)+n_{q}_{ᵢ}) where n_{ƒ}_{ᵢ} and n_{ƒ}_{ᵢ} are quantigraphic and imaging noise processes. Determining an estimate of the photo-quantity requires knowledge of ƒ⁻¹. Then we can write \hat{q}ᵢ(x)=ƒ⁻¹(ƒᵢ(kᵢq(x)))/kᵢ. These estimates are then combined by using a weighted sum to produce a single estimate \hat{q}(x) of the photoquantity present in the original scene at location x. Note that omitting x indicates the entire spatial domain.

Our approach for creating an HDR image from N input LDR images begins with constructing a notional N-dimensional inverse CRF, that incorporates the different exposure and weighting values between the input images. Then we could use this to estimate the photoquantity \hat{q} at each point by writing \hat{q}(x)=ƒ⁻¹(ƒ₁, ƒ₂, . . . , ƒ_{N})/k₁. In this case ƒ⁻¹ is a joint estimator that could be implemented for fast evaluation as an N-dimensional LUT. Recognizing the impracticality of an N-dimensional LUT for large N, we consider pairwise recursive estimation for larger N values in the next paragraph. The joint estimator ƒ⁻¹(ƒ₁, ƒ₂, . . . , ƒ_{N}) may be referred to more precisely as a comparametric inverse camera response function since it always has the domain of a comparagram and the range of the inverse of the response function of the camera under consideration.

Let us assume we have N LDR images that are a constant change in exposure apart, so that ΔEV=log₂ kᵢ₊₁−log₂ kᵢ is a positive constant ∀ iε{1, . . . , N−1}. Now consider specializing to the case N=2 so we have two exposures, one at k₁=1 (without loss of generality, since exposures only have meaning in proportion to one another) and the other at k₂=k. Our estimate of the photoquantity may then be written as \hat{q}(x)=ƒ_{ΔEV}⁻¹(ƒ₁, ƒ₂), where ΔEV=log₂ k.

To apply this pairwise estimator to 3 input LDR images, each with a constant difference in exposure between them, we can proceed by writing

ƒ(\hat{q})=ƒ(ƒ_{ΔEV}⁻¹(ƒ(ƒ_{ΔEV}⁻¹(ƒ₁,ƒ₂)),ƒ(ƒ_{ΔEV}⁻¹(ƒ₂,ƒ₃)))).

In this expression, we first estimate the photoquantity based on images 1 and 2, and then the photoquantity based on images 2 and 3, then these estimates are combined using the same joint estimator, by first putting each of the earlier round (or “level”) of estimates through a virtual camera ƒ, which is the camera response function.

This process may be expanded to any number N of input LDR images, using the recursive relation

ƒᵢ^{(j+1)}=ƒ(ƒ_{ΔEV}⁻¹(ƒᵢ^{(j)},ƒᵢ₊₁^{(j)}))

where j=1, . . . , N−1, i=1, . . . , N−j, and ƒ₁^{(N)} is the final output image, and in the base case, ƒᵢ⁽¹⁾ is the i-th input image. This recursive process may be understood graphically as in FIG. 4 This process forms a graph with estimates of photoquantities as the nodes, and comparametric mappings between the nodes as the edges.

For efficient implementation, rather than computing at runtime or storing values of ƒ⁻¹(ƒ₁, ƒ₂) we can store ƒ(ƒ⁻¹(ƒ₁, ƒ₂)). We call this the comparametric camera response function (CCRF). It is the comparametric inverse CRF evaluated at (or “imaged” through, since we are in effect using a virtual camera) the camera response function ƒ. This means at runtime we require N(N−1)/2 recursive lookups, and we can perform all pairwise comparisons at each level in parallel, where a level is a row of FIG. 4 The reason we can use the same CCRF throughout is due to the fact that each virtual comparametric camera ƒ ∘ƒ⁻¹ returns an exposure that is at the same exposure point as the less-exposed of the two input images (recall that we set k₁=1), so the ΔEV between images remains constant at each subsequent level.

The memory required to store the entire pyramid including the source images is N(N+1) times the amount of memory needed to store a single uncompressed source image with floating-point pixels. Multichannel estimation, for example for color images, can be done by using separate response functions for each channel, at a cost in compute operations and memory storage that is proportional to the number of channels.

To create a CCRF ƒ ∘ƒ⁻¹(ƒ₁, ƒ₂, . . . , ƒ_{N}), the ingredients required are a camera response function ƒ(q), and an algorithm for creating an estimate \hat{q} of photoquantity by combining multiple measurements. Once these have been selected, ƒ ∘ƒ⁻¹ is the camera response evaluated at the output of the joint estimator, and is a function of 2 or more tonal inputs ƒᵢ.

To create a LUT means sampling through the possible tonal values, so for example, to create a 1024×1024 LUT we could execute our \hat{q} estimation algorithm for all combinations of ƒ₁, ƒ₂ε{0, 1/1023, 1/1023, . . . , 1} and store the result of ƒ(\hat{q}) in a matrix indexed by [1023ƒ₁, 1023ƒ₂], assuming zero-based array indexing. Intermediate values may be estimated using linear or other interpolation.

Comparametric image composition, as described here, works with any camera response function model that depends only on the photoquantity, and any compositing algorithm that depends only on the tonal values (e.g., spatial information is excluded).

Explicit construction of the CCRF allows photometric invariants to be analyzed directly.

In the common situation that there is a single camera capturing images in sequence, it is easy to perform updates of the final composited image incrementally, using partial updates, by only updating the buffers dependent on the new input.

We now describe a simple joint photoquantity estimator, using non-linear optimization to compute a CCRF. This method executes in realtime for HDR video, using pairwise comparametric image compositing (see, for example, FIG. 4).

We disclose a simple method for estimating a CCRF. Our first step is to estimate the camera modelparameters; however, any camera model with good empirical fit may be used with this method.

Let scalars ƒ₁ and ƒ₂ form a Wyckoff set from a camera with zero-mean Gaussian noise, and let random variables Xᵢ=ƒᵢ−ƒ(kᵢq), iε{1, 2} be the difference between observation and model, with k₁=1 and k₂=k.

The variances of Xᵢ can be estimated from the inter-quartile range (IQR) along each row and column of the comparagram with the ΔEV of interest (i.e. using the “fatness” of the comparagram). A robust statistical formula, based on the quartiles of the normal distribution, gives \hat{σ}≈IQR/1.349. Discontinuities in \hat{σ}_{X}_{ᵢ} with respect to ƒᵢ can be mitigated by Gaussian blurring of the sample statistics. Using interpolation between samples of the standard deviation, and extrapolation beyond the first and last samples, we can estimate for any value of ƒ₁ or ƒ₂ the corresponding constant σ_{X}_{₁} or σ_{X}_{₂}.

Although we discuss the pairwise N=2 case here, the generalization to N-wise estimation is straightforward.

The probability of \hat{q}, given ƒ₁ and ƒ₂, is

$$\begin{matrix}P ( q = \hat{q} \vert {f}_{1} , {f}_{2} ) = \frac{P ( q ) P ( {f}_{1} \vert q ) P ( {f}_{2} \vert q )}{P ( {f}_{1} \vert {f}_{2} )} \\ = \frac{P ( q ) P ( {f}_{1} \vert q ) P ( {f}_{2} \vert q )}{{\int }_{0}^{∞} P ( {f}_{1} \vert q ) P ( {f}_{2} \vert q ) d q} ∝ \\ P ( q = \hat{q} ) P ( {f}_{1} \vert q ) P ( {f}_{2} \vert q ) .\end{matrix}$$

For simplicity, we choose a uniform prior, which gives us Pₚᵣᵢₒᵣ(q=\hat{q})=CONSTANT. Using Xᵢ, we have

$$\begin{matrix}{P}_{model} ( {f}_{i} \vert q ) = Normal ( {μ}_{{X}_{i}} = 0 , {σ}_{{X}_{i}}^{2} ) \\ = \frac{1}{\sqrt{2 π} {σ}_{{X}_{i}}} exp [ - \frac{{( x - {μ}_{{X}_{i}} )}^{2}}{2 {σ}_{{X}_{i}}^{2}} ] \\ = \frac{1}{\sqrt{2 π} {σ}_{{X}_{i}}} exp [ - \frac{{( {f}_{i} - f ( {k}_{i} q ) )}^{2}}{2 {σ}_{{X}_{i}}^{2}} ] .\end{matrix}$$

To maximize P(q=\hat{q}|ƒ₁, ƒ₂) with respect to q, we remove constant factors and equivalently minimize −log(P). Then the optimal value of q, given ƒ₁ and ƒ₂, is

$$q = {argmin}_{q} [ \frac{{( {f}_{1} - f ( q ) )}^{2}}{{σ}_{{X}_{1}}^{2}} + \frac{{( {f}_{2} - f ( kq ) )}^{2}}{{σ}_{{X}_{2}}^{2}} ] .$$

where qε[0, ∞), and ƒ(q) is the camera response function model. In practice good estimates of optimal q values can be found using, for example, the LevenbergMarquardt algorithm.

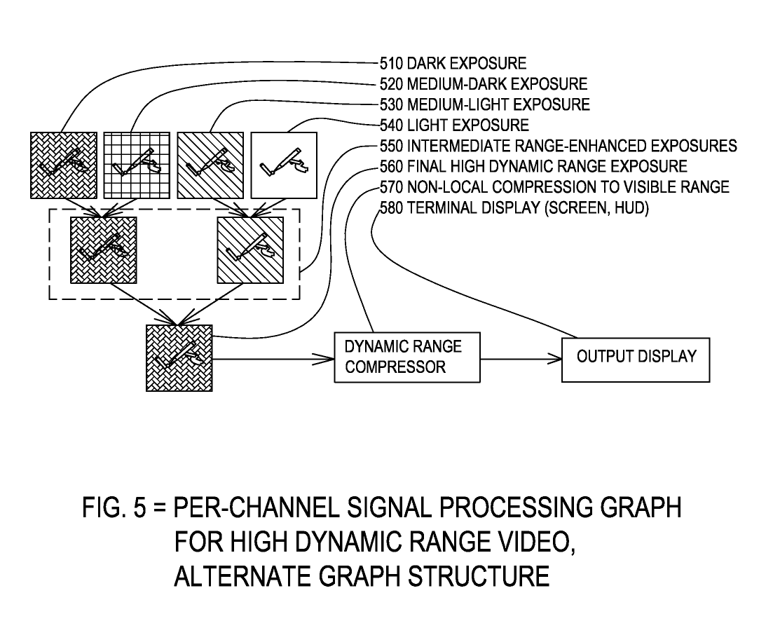

FIG. 5 depicts an alternate embodiment of the signal processing graph for processing LDR images that is directly applicable when the number of input images is a power of two. This structure requires twice the amount of computer memory for lookup tables as the structure in FIG. 4, but only half the number of lookups, assuming the input images have an equal change in exposure value. This implementation demonstrates a tradeoff between memory requirements and exectution speed. For arbitrary larger numbers of input images, combining this approach with that of FIG. 4 enables the number of lookups to scale linearly with the number of input images.

With reference to FIG. 5, we denote the contents of 510 as ƒ₁, 520 as ƒ₂, 530 as ƒ₃, 540 as ƒ₄, and 560 as ƒ(\hat{q}) in the following equations.

The following form for image compositing in the case of four input (N=4) is shown in FIG. 5.

ƒ(\hat{q})=ƒ(ƒ_{2ΔEV}⁻¹(ƒ(ƒ₁,ƒ₂)),ƒ(ƒ_{ΔEV}⁻¹(ƒ₃,ƒ₄))))

in which case we only perform 3 lookups at runtime, instead of 6 using the previous structure. However, we must store twice as much lookup information in memory: for ƒ ∘ƒ_{ΔEV}⁻¹ as before, and for ƒ ∘ƒ_{2ΔEV}⁻¹, since the results of the inner expressions are no longer ΔEV apart, but instead are twice as far apart in exposure value, 2ΔEV, as shown in FIG. 5.
As a recursive relation for N=2ⁿ, nεN we have

ƒᵢ^{(j+1)}=ƒ(ƒ_{jΔEV}⁻¹(ƒ₂ᵢ₋₁^{(j)},ƒ₂ᵢ^{(j)}))

where j=1, . . . , log₂N, and i=1, . . . , N/2^{j-1}. The final output image is ƒ₁^{(log}^{₂}^{N+1)}, and ƒᵢ⁽¹⁾ is the i-th input image. This form requires N−1 lookups. In general, by combining this approach with the previous graph structure it can be seen that comparametric image composition can always be done in O(N) lookups for any number of (N) input low dynamic range signals.

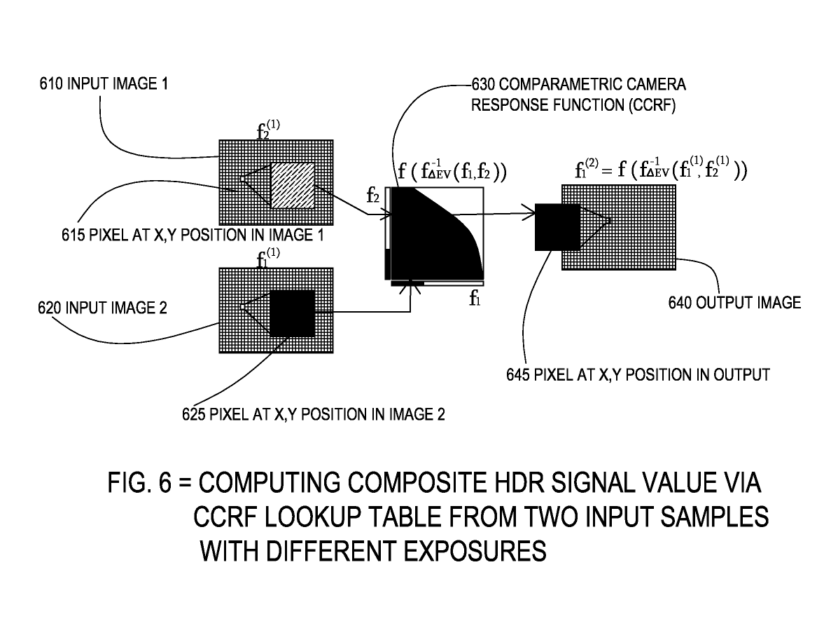

FIG. 6 depicts a signal processing algorithm of the electric seeing aid, as applied to two differently exposed images of approximately the same subject matter. In FIG. 6 the core high dynamic range estimation process is shown pictorially for the example of two differently exposed images, although the process for any signals and sensors of limited dynamic range is analogous.

This is the process that takes place in each pairwise comparison depicted in FIG. 4 and FIG. 5. For any two inputs, this process computes an output which is later composited with new information, or sent for display. While pairwise compositing is preferred for computational tractability, the process may be easily extended to higher numbers of simultaneous inputs, depending on the structure of the computer's memory system to maintain high throughput.

The first input image (signal) 610 is considered using single sample 615, in combination with the corresponding sample 625 from the second input 620. The values of samples 615 and 625 are used to index the lookup table 630, where these values may be floating-point indices using an interpolant to provide intermediate values of the lookup table (joint response function) 630. The result of the compositing process is then stored at the corresponding location 645 of the output image (signal) 640.

From the foregoing description, it will thus be evident that the present invention provides a design for a system to help a person see, and possibly also hear better, while enganged in a light (or sound) producing activity. As various changes can be made in the above embodiments and operating methods without departing from the spirit or scope of the invention, it is intended that all matter contained in the above description or shown in the accompanying drawings should be interpreted as illustrative and not in a limiting sense.

Variations or modifications to the design and construction of this invention, within the scope of the invention, may occur to those skilled in the art upon reviewing the disclosure herein. Such variations or modifications, if within the spirit of this invention, are intended to be encompassed within the scope of any claims to patent protection issuing upon this invention.

What I claim is:

1. A seeing aid to help a user of an electric arc welding process see aspects of the process or the environment around the process, said seeing aid comprising:
a weld sensor that senses when welding is taking place;
an illumination modulator for a worklight illuminating a work area of said electric arc welding process,
said illumination modulator varying a quantity of illumination in response to said weld sensor.

2. The seeing aid of claim 1, said seeing aid further including:
a viewing device;
a worklight connected to said illumination modulator;
where said viewing device includes said weld sensor, said viewing device darkening in response to an electric arc of said electric arc welding process, and said viewing device lightening in response to an increase in illumination of said worklight, and darkening in response to a decrease in illumination of said worklight.

3. The seeing aid of claim 1, further including an arc modulator for said electric arc, said arc modulator for modulating said arc in an orthogonal or opposing lightspace to said illumination modulator.

4. The seeing aid of claim 1, said seeing aid further including:
a controller,
said controller for increasing light output of a worklight when said welding has begun.

5. The seeing aid of claim 4 where said light modulation is by way of a relay to turn on said worklight when said welding has begun, and to turn off said worklight when said welding has finished.

6. The seeing aid of claim 4 where said sensing is by way of an auto-darkening welding helmet, and said light modulation is by way of switching to turn on or up said worklight when said welding helmet is in a darkend state, and to turn off or dim down said worklight when said welding helmet is in a lightened state.

7. The seeing aid of claim 4 where said seeing aid further includes a headworn viewing aid to help a user of a light-producing activity sense aspects of the process or the environment around the process, said seeing aid further including:
a worklight illuminating a work area of said light-producing activity;
said seeing aid more responsive to light from said worklight than from light from said light-producing activity.

8. An auto-darkening welding helmet using the vision improvement system of claim 7, where said worklight produces a periodically varying level of light, and said helmet darkens less when said worklight produces more light, and said helmet darkens more when said worklight produces less light.

9. An auto-darkening welding helmet using the vision improvement system of claim 7, where said worklight is a pulsating strobe light, said vision improvement system also including a processor, said processor issuing a control signal to said helmet to lighten in synchronization with a light output of said strobe light.

10. The auto-darkening welding helmet of claim 9, said helmet having a user-adjustable control to set the relative lightspace proportion between light due to the light producing process and light due to the worklight.

11. An electric seeing aid, said seeing aid comprising a mirrorshade, said mirror-shade having an outward-facing mirrored surface on an outward side of a low-transmissivity transparent material, said seeing aid having a camera arranged for receiving reflected light from said mirrorshade, said seeing aid also having a display device responsive to an output from said camera.

12. The electric seeing aid of claim 11, said electric seeing aid including:
a wearable computer;
physiological sensors;
said helmet for operably controlling a welding power supply in response to an output from said physiological sensors.

13. The seeing aid of claim 12 where said physiological sensor is a brainwave sensor, and where said power supply increases output when both the Alpha and Beta wave input of the brainwave sensor increase together.

14. The seeing aid of claim 12 where said power supply pulsates at the same rate as a heartbeat of a user of said seeing aid.

15. An electric seeing aid said electric seeing aid comprising a camera that captures a plurality of differently exposed images of approximately identical subject matter, said seeing aid also including a processor, said processor computing an HDR (High Dynamic Range) image of said subject matter, said processor updating said HDR image each time a new exposure is captured.

16. The electric seeing aid of claim 15 said electric seeing aid comprising a video camera that captures alternately at least two images, one of lesser exposure, and one of greater exposure, said processor generating an output image each time a new input image is captured.

17. The electric seeing aid of claim 15, said processor implementing a process for combing a number, N, of multiple images from said camera, said process comprising the steps of indexing into an N dimensional LUT (lookup table), for each output pixel at coordinates corresponding to each of the input images, where the output pixel value is given by the LUT evaluated at the indicies given by the pixel values in the input images.

18. The electric seeing aid of claim 15, said processor implementing a process for combing at least two differently exposed pictures of the same subject matter, said processor executing a process wherein a pixel value of an output image is responsive to an output of an element of a two-dimensional lookup table, said element being indexed by the pixel values of the two input images.
